(19) United States
(12) Patent Application Publication
(10) Pub. No.: US 2005/0004106 A1
(43) Pub. Date: Jan. 6, 2005

(54) Combinations of GABA modulators and anticonvulsants, and atypical antipsychotics

(75) Inventor: Steven Joseph Romano, New York, NY (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/845,826

(22) Filed: May 14, 2004

Related U.S. Application Data
(60) Provisional application No. 60/471,188, filed on May 16, 2003.

(51) Int. Cl. A61K 31/5513; A61K 31/519

(52) U.S. Cl. 514/220; 514/221; 514/259.41; 514/389; 514/290; 514/217

(57) ABSTRACT

This invention relates to combinations of an atypical antipsychotic, and a GABA modulator, a benzodiazepine, and/or an anticonvulsant drug, kits containing such combinations, pharmaceutical compositions comprising such combinations, and methods of using such combinations to treat patients suffering from treatment-resistant anxiety disorders, psychotic disorders or conditions, or mood disorders or conditions.

This application claims priority under 35 U.S.C 119 of U.S. Provisional 60/471,188 filed May 16, 2003. The entire contents of the prior application are incorporated herein by reference.

### FIELD OF THE INVENTION

The present invention relates to pharmaceutical compositions comprising combinations of ziprasidone, a prodrug thereof or a pharmaceutically acceptable salt of ziprasidone or said prodrug and a GABA modulator, a prodrug thereof or a pharmaceutically acceptable salt of a GABA modulator or said prodrug, or an anticonvulsant drug, a prodrug thereof or a pharmaceutically acceptable salt of an anticonvulsant drug or said prodrug and/or a benzodiazepine, a prodrug thereof or a pharmaceutically acceptable salt of a benzodiazepine or said prodrug, kits containing such combinations and methods of using such combinations to treat patients, including humans, suffering from treatment resistant anxiety disorders, psychotic disorders or conditions, or mood disorders or conditions. This invention also relates to additive and synergistic combinations of ziprasidone, a prodrug thereof or a pharmaceutically acceptable salt of ziprasidone and a GABA modulator, a prodrug thereof or a pharmaceutically acceptable salt of said GABA modulator or said prodrug, whereby those additive and synergistic combinations are useful in treating patients, including humans, suffering from treatment-resistant anxiety disorders, psychotic disorders or conditions, and/or mood disorders or conditions.

### BACKGROUND OF THE INVENTION

Schizophrenia is a common and serious mental disorder characterized by loss of contact with reality (psychosis), hallucinations (false perceptions), delusions (false beliefs), abnormal thinking, flattened affect, diminished motivation, and disturbed work and social functioning.

Atypical antipsychotics offer several clinical benefits over the conventional antipsychotics, which were the mainstays of care until the past decade. The principal mechanism underlying the many clinical benefits of the atypical agents is their ability to separate the antipsychotic effect from the extrapyramidal side effect (EPS). The distinct advantages over traditional antipsychotic medications include greater improvement in negative and cognitive symptoms, better antidepressant and mood stabilization effects, lower risk of parkinsonian side effects and tardive dyskinesia, and greater efficacy in otherwise refractory or treatment-resistant patients.

The clinical profile of the atypical and conventional antipsychotics can be understood in terms of their different pharmacological profiles. The conventional antipsychotics are antagonists of dopamine (D₂) receptors. The atypical antipsychotics also have D₂ antagonistic properties, but possess different binding kinetics to these receptors and activity at other receptors, particularly 5-HT_{2A, 5}-HT_{2c}, and 5-HT_{1D} (Schmidt B et al, Soc. Neurosci. Abstr. 24:2177, 1998).

The class of atypical antipsychotics includes clozapine (clozaril®), risperidone (risperdal®), olanzapine (zyprexa®), quetiapine (seroquel®), aripiprazole (abilify®) and ziprasidone (geodon®). Ziprasidone is an atypical antipsychotic whose efficacy in the treatment of schizophrenia has been examined in an extensive clinical trial program that includes both short term and long term studies. Ziprasidone is indicated for the treatment of schizophrenia or psychotic disorders and is widely used in a variety of mood disorders, psychiatric medical syndromes and severe personality disorders.

Commonly assigned U.S. Pat. Nos. 4,831,031, 4,883,795, 6,245,766 and 6,126,373, which are hereby incorporated by reference, each disclose that ziprasidone has utility in the treatment of treatment-resistant anxiety disorders, psychotic disorders, and mood disorders.

The term “ziprasidone”, as used herein, unless otherwise indicated, encompasses the free base of the compound ziprasidone and all pharmaceutically acceptable salts thereof.

GABA is the major inhibitory neurotransmitter in the patient in the central nervous system (CNS). GABA receptors can be found in 60-80% of CNS neurons. <Allosteric facilitation of GABA receptors occurs at several distinct sites; the compounds which bind there are used as sedatives and anxiolytics.

GABA modulators have been disclosed to be useful in antiseizure therapy for central nervous system disorders such as epilepsy, Huntington's chorea, cerebral ischemia, Parkinson's disease, tardive dyskinesia and spasticity. GABA agonists have also been disclosed to be useful antidepressants, anxiolytics and antipsychotics.

Commonly assigned U.S. Pat. No. 4,024,175, which is hereby incorporated by reference, discloses that GABA modulators have utility in the treatment of treatment-resistant anxiety, psychotic disorders and conditions, and mood disorders and conditions.

GABA modulators well known in the art include muscimol, progabide, riluzole, baclofen, gabapentin (Neurontin®), vigabatrin, valproic acid, (Depakene®, Depakote®) tiagabine (Gabitril®), lamotrigine (Lamictal®), pregabalin, topiramate (Topamax®) and analogs, derivatives, prodrugs and pharmaceutically acceptable salts of those GABA modulators.

Benzodiazepines have been used for several decades in connection with a broad spectrum of diseases. The major known effects of benzodiazepines are anticonvulsant, muscle relaxing, sedative, hypnotic, anxiolytic, and antipsychotic. The mechanism underlying the effect of the benzodiazepine drugs is unknown but it is believed to relate to the GABA system of the CNS.

When any of the anxiolytic or antipsychotic effects are desired, it is often a problem that the sedative and hypnotic effects of benzodiazepines prohibit the use of high dosages of benzodiazepines, or, when such high dosages are nevertheless necessary to get a reasonable effect of the treatment, make it necessary to hospitalize the patient. Even in the dosages used against disorders or conditions, e.g. anxiety, the sedative effect of benzodiazepines may be disadvantageous.

According to DSM-IV, Generalized Anxiety Disorder is characterized by persistent and excessive anxiety and worry about a number of events and activities occurring on more days than not, for at least 6 months. Anxiety disorders are the most common form of mental illness in the United States affecting more than 19 million adults yearly. Treatments for anxiety disorders include the Selective Serotonin Reuptake Inhibitors (SSRIs), buspirone, venlafaxine and benzodiazepines. Typical and atypical antipsychotics investigated as therapeutic agents with utility for anxiety have demonstrated a more tolerable side effect profile, and a lower incidence of tardive dyskinesia. The serotoninergic properties of ziprasidone may make it useful in the treatment of anxiety disorders.

There remains a low rate of complete remission reported with benzodiazepines and antidepressants, thereby warranting alternative augmentation strategies to reduce disability and suffering in this chronic disorder.

Posttraumatic stress disorder (PTSD) is a severe and often chronic mental illness. PTSD has lifetime population prevalence of about 10% in the U.S., making it among the most prevalent of psychiatric disorders. The most common traumatic stressors are rape, domestic violence, child abuse, assault, accidents, and disasters. PTSD is characterized by symptoms in three clusters, intrusive, avoidant, and arousal. The intrusive symptom cluster (flashbacks, nightmares, intrusive thoughts, physiological and psychological arousal upon reminders of trauma) is considered unique to PTSD, and is not seen in any other psychiatric condition. Though classified as an anxiety disorder in DSM-IV, PTSD is accompanied by psychotic symptoms in almost half of patients. Treatment consists of the Selective Serotonin Reuptake Inhibitors (SSRIs) such as sertraline, GABA modulators, and benzodiazepines. The psychotic symptoms are treated as add-on therapy with antipsychotic agents. Therefore, a combination product would have utility in this patient population.

Mood disorders, also known as affective disorders, are a group of heterogeneous, typically recurrent illnesses including unipolar (depressive) and bipolar (manic-depressive) disorders, dysthymic disorder, and cyclothymic disorder that are characterized by pervasive mood disturbances, psychomotor dysfunction, and vegetative symptoms. Mood disorders may affect 20% of women and 12% of men during their lifetime. They are the most prevalent of psychiatric disorders, accounting for as many as 65% of psychiatric outpatients, and 10% of all patients seen in nonpsychiatric medical settings (The Merck Manual, 17^{th} ed., Merck & Co. 1999, p. 1526).

Lithium, the standard of care for mood disorder has a response rate of only 50% and is associated with side effects. Anticonvulsants have been used in mood disorders as mood stabilizers and are indicated for use in bipolar disorders. For example, valproic acid and derivatives, e.g. divalproex sodium or carbamazepine at doses of 500 to 2000 mg daily have shown limited efficacy. Antipsychotic agents are also clinically used in this patient population. A combination product containing anticonvulsants and atypical antipsychotics will have significant utility in the treatment of these patients.

Mental illness is particularly difficult to treat in that not all patients react similarly to the same treatment regimen. Patients often require multiple drug therapies. There also exists a large number of untreated individuals and treatment-resistant patients in need of effective therapy.

Exacerbating this is the problem of patient noncompliance. For example, it is conventionally thought that substantial numbers of patients with mental illnesses are not or only partially compliant with their medication. Poor compliance can cause relapses thereby negating whatever benefits were achieved through treatment in the first place.

Simplification of the regimen by combining several therapeutic agents, reduces the opportunity for patient noncompliance as occurs with a more rigorous schedule. There is a need for pharmaceutical combinations and pharmaceutical kits which employ atypical antipsychotics efficacious for the treatment of, e.g. treatment-resistant anxiety, psychotic disorders and conditions and mood disorders.

The present invention is directed to compositions which reduce or overcome these disadvantages in novel pharmaceutical combinations of ziprasidone and GABA modulators, anticonvulsants and benzodiazepines for the treatment of treatment-resistant anxiety, psychotic disorders and symptoms, and mood disorders and conditions.

### SUMMARY OF THE INVENTION

The present invention is directed to pharmaceutical compositions, therapeutic methods of treatment, and kits which employ an atypical antipsychotic together with a GABA modulator, an anticonvulsant or a benzodiazepine.

According to the invention, it has surprisingly been found that the pharmaceutical combinations of the present invention can provide synergistic and additive effects with less side effects and a reduction in use of concomitant psychotropic medications such as antidepressants, sedatives and mood stabilizers such as lithium.

Thus according to one aspect, the present invention provides a combination of an atypical antipsychotic agent and a GABA modulator, or an anticonvulsant or a benzodiazepine. Atypical antipsychotics which may be used in the present invention include olanazapine, clozapine, risperidone, sertindole, quetiapine, aripiprazole, amisulpride and ziprasidone. In general, pharmaceutical combinations and methods of treatment using ziprasidone as the first therapeutic agent are preferred.

A further feature of the present invention is a method of reducing the amount of the atypical antipsychotic agent required to produce an anti-anxiety, antipsychotic and mood stabilizing effect which comprises treating a patient with a therapeutically effective amount of a drug combination according to the present invention.

It is also a feature of this invention that the use of such drug combinations will enhance the effect of the atypical antipsychotic agent to be used and therefore allow reduced quantities of the antipsychotic agent to be used and, therefore allow better management of drug-related toxicity and side effects.

The invention offers advantages over previous methods for treating neuropsychiatric disorders. For example, in the method of treatment of the present invention, the atypical antipsychotic counteracts the typical sedative and hypnotic effects of the benzodiazepine. Other features and advantages of the invention will be apparent from the following detailed description and from the claims.

### DETAILED DESCRIPTION OF THE INVENTION

The present invention is directed to pharmaceutical compositions comprising: an amount of ziprasidone, a prodrug thereof or a pharmaceutically acceptable salt of ziprasidone or said prodrug; and an amount of a GABA modulator, an anticonvulsant drug and/or a benzodiazepine, prodrugs thereof or pharmaceutically acceptable salts of said GABA modulator, anticonvulsant drug or benzodiazepine; and a pharmaceutically acceptable vehicle, carrier or diluent.

The present invention is directed to a therapeutic method and pharmaceutical compositions comprising ziprasidone and a GABA modulator useful for treating treatment-resistant anxiety disorders; ziprasidone and an anticonvulsant drug useful in the treatment of mood disorders or psychotic disorders or treatment; and ziprasidone and a benzodiazepine effective in the treatment of treatment-resistant anxiety and/or psychotic disorders or conditions.

The present invention is also directed to a therapeutic method and a pharmaceutical composition comprising ziprasidone and a GABA modulator useful for treatment of treatment-resistant anxiety disorders.

The present invention is further directed to a therapeutic method and a pharmaceutical composition comprising ziprasidone and a benzodiazepine useful for treatment of psychotic disorders or conditions or treatment-resistant anxiety disorders.

The present invention is still further directed to a therapeutic method and a pharmaceutical composition comprising ziprasidone and an anticonvulsant drug useful for treating mood disorders or conditions, psychotic disorders or conditions, or psychotic symptoms.

This invention is also directed to kits for achieving a therapeutic effect in a patient comprising an amount of ziprasidone, a prodrug thereof or a pharmaceutically acceptable salt of said ziprasidone and a pharmaceutically acceptable vehicle, carrier or diluent in a first unit dosage form; and an amount of a GABA modulator or an anticonvulsant drug or a benzodiazepine, prodrugs thereof or pharmaceutically acceptable salts of said GABA modulator, anticonvulsant drug or benzodiazepine and a pharmaceutically acceptable vehicle, carrier or diluent in a second unit dosage form and a container.

This invention is also directed to methods of treating a patient in need of therapy comprising administering to said patient an amount of a first drug, the first drug being ziprasidone, a prodrug thereof or a pharmaceutically acceptable salt of ziprasidone, and an amount of a second compound, the second compound being a GABA modulator, an anticonvulsant drug or a benzodiazepine, a prodrug thereof or a pharmaceutically acceptable salt of the GABA modulator, anticonvulsant drug or benzodiazepine.

This invention is further directed to methods for treating a patient in need of therapy comprising administering to said patient
an amount of a first compound, the first compound being ziprasidone, a prodrug thereof or a pharmaceutically acceptable salt of ziprasidone or the prodrug; and
an amount of a second compound, the second compound being a GABA modulator, an anticonvulsant drug or a benzodiazepine, a prodrug thereof or a pharmaceutically acceptable salt of the GABA modulator, anticonvulsant drug or benzodiazepine or said prodrug;
wherein said first compound and said second compound are each optionally and independently administered together with a pharmaceutically acceptable vehicle, carrier or diluent.

This invention is also directed to methods for treating a patient in need of therapy comprising administering to the patient a pharmaceutical composition comprising
a) an amount of a first compound, the first compound being ziprasidone, a pharmaceutically salt of ziprasidone, a prodrug of ziprasidone, or a pharmaceutically acceptable salt of a ziprasidone prodrug; and
b) an amount of a second compound, the second compound being a GABA modulator, an anticonvulsant drug, a benzodiazepine, a prodrug thereof or a pharmaceutically acceptable salt of the GABA modulator, or anticonvulsant drug, or benzodiazepine or the prodrug; and, optionally,
a pharmaceutically acceptable vehicle, carrier or diluent.

The methods of this invention include therapeutic treatment of treatment-resistant anxiety. Treatment-resistant anxiety which may be treated by the methods of this invention includes, inter alia, treatment-resistant obsessive compulsive disorder or treatment-resistant post-traumatic stress disorder.

The methods of this invention include therapeutic treatment of psychotic disorders or conditions. Psychotic disorders which can be treated by the methods of this invention include, inter alia, schizophrenia, schizophreniform disorder, schizoaffective disorder, delusional disorder, brief psychotic disorder, shared psychotic disorder.

The methods of this invention include therapeutic treatment of mood disorders or conditions. Mood disorders are a group of heterogeneous illnesses including unipolar (depressive) and bipolar (manic-depressive) disorders that are characterized by pervasive mood disturbances, psychomotor dysfunction, and vegetative symptoms. While depression and elation are the core affective components, anxiety and irritability are equally common, explaining the continued popularity of the broader rubric “affective disorders”, the previous official designation.

Preferred GABA modulators for use in the combinations, pharmaceutical compositions, methods and kits of this invention include: muscimol, progabide, riluzole, baclofen, gabapentin (Neurontin®), vigabatrin, valproic acid, tiagabine (Gabitril®), lamotrigine (Lamictal®), pregabalin, phenyloin (Dilantin®), carbamazepine (Tegretol®), topiramate (Topamax®), prodrugs thereof and pharmaceutically acceptable salts of the GABA modulators and the prodrugs.

More preferred GABA modulators for use in the combinations, pharmaceutical compositions, methods and kits of this invention include gabapentin, tiagabine, lamotrigine, topiramate, pregabalin, prodrugs thereof and pharmaceutically acceptable salts of the GABA modulators and the prodrugs.

A particularly preferred GABA modulator for use in the combinations, pharmaceutical compositions, methods and kits of this invention is pregabalin, a prodrug thereof or a pharmaceutically acceptable salt of pregabalin or a prodrug thereof.

Another particularly preferred GABA modulator for use in the combinations, pharmaceutical compositions, methods and kits of this invention is gabapentin (Neurontin®), a prodrug thereof or a pharmaceutically acceptable salt of gabapentin (Neurontin®) or prodrug thereof.

Preferred anticonvulsants for use in the combinations, pharmaceutical compositions, methods and kits of this invention include: hydantoins such as phenyloin (Dilantin®), mephenyloin (Mesantoin®); succinimides such as ethosuximide (Zarontin®), oxazolidinediones such as trimethadione (Tridione®), carbamazepine (Tegretol®), primadone (Mysoline®), valproic acid (Depakote®), prodrugs thereof and pharmaceutically acceptable salts of the anticonvulsants and prodrugs thereof.

More preferred anticonvulsants for use in the combinations, pharmaceutical compositions, methods and kits of this invention include phenyloin and valproic acid, prodrugs thereof and pharmaceutically acceptable salts of the anticonvulsants and prodrugs thereof, and pharmaceutically acceptable salts of the prodrugs.

A particularly preferred anticonvulsant for use in the combinations, pharmaceutical compositions, methods and kits of this invention is valproic acid, a prodrug thereof or a pharmaceutically acceptable salt of valproic acid or prodrug thereof.

Another particularly preferred anticonvulsant for use in the combinations, pharmaceutical compositions, methods and kits of this invention is phenyloin, a prodrug thereof or a pharmaceutically acceptable salt of phenyloin or prodrug thereof.

Preferred benzodiazepines for use in the combinations, pharmaceutical compositions, methods and kits of this invention include: alprazolam, chlordiazepoxide, clonazepam, clorazepate, diazepam, halazepam, lorazepam, temazepam and oxaxepam, prodrugs thereof and pharmaceutically acceptable salts of benzodiazepines and prodrugs thereof.

More preferred benzodiazepines for the use in combinations, pharmaceutical compositions, methods and kits of this invention include clonazepam, diazepam and lorazepam, prodrugs thereof and pharmaceutically acceptable salts of anticonvulsants and prodrugs thereof.

A particularly preferred benzodiazepine for the use in combinations, pharmaceutical compositions, methods and kits of this invention is clonazepam, a prodrug thereof or a pharmaceutically acceptable salt of clonazepam or a prodrug thereof.

Another particularly preferred benzodiazepine for the use in combinations, pharmaceutical compositions, methods and kits of this invention is lorazepam, a prodrug thereof or a pharmaceutically acceptable salt of lorazepam or a prodrug thereof.

The combinations of this invention comprise at least two active components: ziprasidone, a prodrug thereof or a pharmaceutically acceptable salt, and a GABA modulator, a prodrug thereof or a pharmaceutically acceptable salt of the GABA modulator; or ziprasidone, a prodrug thereof or a pharmaceutically acceptable salt, and an anticonvulsant, a prodrug thereof or a pharmaceutically acceptable salt of an anticonvulsant or a prodrug; or ziprasidone, a prodrug or pharmaceutically acceptable salt and a benzodiazepine, a prodrug thereof or a pharmaceutically acceptable salt of a benzodiazepine. The combinations of this invention include a pharmaceutically acceptable vehicle, carrier or diluent.

The combinations result in synergistic action allowing a lower dose of the atypical antipsychotic to be administered while achieving the same psychotropic effect. The dosage of the atypical antipsychotic may be reduced by about 25-90%, for example, about 40-80% and typically about 50-70%. The reduction in amount of antipsychotic required will be dependent on the amount of second therapeutic agent given.

The selection of the dosage of the first and second therapeutic agents is that which can provide relief to the patient as measured by a reduction or amelioration of symptoms associated with the disorder or condition of the patient. As is well known, the dosage of each component depends on several factors such as the potency of the selected specific compound, the mode of administration, the age and weight of the patient, the severity of the condition to be treated, and the like. This is considered to be within the skill of the artisan and one can review the existing literature regarding each component to determine optimal dosing. To the extent necessary for completeness, the synthesis of the components of the compositions and dosages are as described in the listed patents or the Physicians' Desk Reference, 57th ed., Thompson, 2003 which are expressly incorporated herein by reference. Desirably, when ziprasidone is selected as the active agent, the daily dose contains from about 5 mg to about 460 mg. More preferably, each dose of the first component contains about 20 mg to about 320 mg of the ziprasidone, and even more preferably, each dose contains from about 20 mg to about 160 mg of zirpasidone. Pediatric dosages may be less. This dosage form permits the full daily dosage to be administered in one or two oral doses, for example.

General outlines of the dosages for the atypical antipsychotics, GABA modulators, anticonvulsants, and benzodiazepines, and some preferred dosages, are provided herein. This list is not intended to be complete but is merely a guideline for any of the desired combinations of the present invention.
Olanzapine: from about 0.25 to about 100 mg, once/day; preferred, from about 1 to about 30 mg, once/day; and most preferably about 1 to about 25 mg once/day;
Clozapine: from about 12.5 to about 900 mg daily; preferred, from about 150 to about 450 mg daily;
Risperidone: from about 0.25 to about 16 mg daily; preferred from about 2-8 mg daily;
Sertindole: from about 0.0001 to about 1.0 mg/kg daily;
Quetiapine: from about 1.0 to about 40 mg/kg given once daily or in divided doses;
Asenapine: from about 0.005 to about 60 mg total per day, given as a single dose or in divided doses;
Carbamezepine: from about 200 to about 1200 mg/day; preferably about 400 mg/day;
Valproic Acid: from about 250 to about 2500 mgday; preferably about 1000 mg/day;
Lamotrigine: from about 50 to about 600 mg/day in 1 to 2 doses; preferably about 200 to about 400 mg; most preferably about 200 mg;
Gabapentin: from about 300 to about 3600 mg/day in 2 to 3 divided doses; preferably 300 to about 1800 mg/day; most preferably about 900 mg/day;
Tiagabine: from about 2 to about 56 mg/day in 2 to 4 divided doses; preferably about 32 to about 56 mg/day; most preferably about 56 mg/day.
Topiramate: from about 200 to about 600 mg/day divided in 2 doses; most preferably about 400 mg/day.

The Table below provides additional dosage ranges:

Drug Name

| Brand name | Generic Name | Dosage Range |
|---|---|---|
| Klonopin | Clonazepam | Minimum: 0.25 mg |
| | | Maximum: 20 mg |
| Tranxene | Clorazepate | Minimum: 3.75 mg. |
| | Dipotassium | Maximum: 60 mg |
| Valium | Diazepam | Minimum: 1 mg. |
| | | Maximum: 40 mg. |
| Xanax | Alprazolam | Minimum: 0.25 mg |
| | | Maximum: 4 mg |
| Gabitril | Tiagabine | Minimum: 4 mg |
| | | Maximum: 56 mg |
| Neurontin | Gabapentin | Minimum: 100 mg |
| | | Maximum: 2400 mg |
| Dilantin | Phenytoin | Minimum: 50 mg |
| | | Maximum: 1200 mg |
| Carbatrol | Carbamazepine | Minimum: 200 mg |
| Capsules | ER | Maximum: 1200 mg |
| Depakote | Valproic acid | Minimum: 250 mg |
| | | Maximum: 2000 mg |
| Felbatol | Felbamate | Minimum: 1200 mg |
| | | Maximum: 3600 mg |
| Keppra | Levetiracetam | Minimum: 1000 mg |
| | | Maximum: 3000 mg |
| Tegretol | Carbamazepine | Minimum: 200 mg |
| | | Maximum: 1200 mg |
| Topamax | Topiramate | Minimum: 25 mg |
| | | Maximum: 400 mg |
| Celontin | Methoximide | Minimum: 150 mg |
| | | Maximum: 1200 mg |
| Trileptal | Oxcarbazepine | Minimum: 300 mg |
| | | Maximum: 2400 mg |
| Zonegran | Zonisamide | Minimum: 100 mg |
| | | Maximum: 600 mg |
| Lamictal | Lamotrigine | Minimum: 200 mg |
| | | Maximum: 400 mg |
| Zarontin Capsules | Ethosuximide | Minimum: 250 mg |
| | | Maximum: 1500 mg |

In more general terms, one would create a drug combination of the present invention by choosing a dosage of first and second component compounds according to the spirit of the above guideline.

The atypical antipsychotics of the present invention are useful in treating schizophrenia, bipolar disorders, and dementia.

The presently preferred atypical antipsychotic used according to the invention is ziprasidone. Ziprasidone (5-[2-[4-(1,2-benzisothiazol-3-yl)piperazin-1-yl]ethyl]-6-chloroindolin-2-one hydrochloride hydrate) is a benzisothiazolyl piperazine-type atypical antipsychotic with in vitro activity as a 5-HT_{1A} receptor agonist and an inhibitor of serotonin and norepinephrine reuptake (See e.g. U.S. Pat. No. 4,831,031). The postsynaptic 5-HT_{1A} receptor has been implicated in both depressive and anxiety disorders (N M Barnes, T Sharp, 38 Neuropharmacology 1083-152,1999). Oral bioavailability of ziprasidone taken with food is approximately 60%, half-life is approximately 6-7 hours, and protein binding is extensive.

Ziprasidone is efficacious for the treatment of patients with schizophrenia and schizomood disorders, refractory schizophrenia, cognitive impairment in schizophrenia, affective and anxiety symptoms associated with schizoaffective disorder and bipolar disorder.

The drug is considered a safe and efficacious atypical antipsychotic (Charles Caley & Chandra Cooper, 36 Ann. Pharmacother. 839-51, 2002).

The present invention is useful in treating mental disorders and conditions, the treatment of which is facilitated by the administration of ziprasidone. Thus, the present invention has application where ziprasidone use is indicated as, e.g., in U.S. Pat. Nos. 6,245,766; 6,245,765; 6,387,904; 5,312,925; 4,831,031; and European EP 0901789 published Mar. 17, 1999, all of which are incorporated herein by reference.

Other atypical antipsychotics which can be used include, but are not limited to: Olanzapine, 2-methyl-4-(4-methyl-1-piperazinyl)-10H-thieno[2,3-b][1,5]benzodiazepine, is a known compound and is described in U.S. Pat. No. 5,229,382 as being useful for the treatment of schizophrenia, schizophreniform disorder, acute mania, mild anxiety states, and psychosis. U.S. Pat. No. 5,229,382 is herein incorporated by reference in its entirety;

Clozapine, 8-chloro-11-(4-methyl-1-piperazinyl)-5H-dibenzo[b,e][1,4]diazepine, is described in U.S. Pat. No. 3,539,573, which is herein incorporated by reference in its entirety. Clinical efficacy in the treatment of schizophrenia is described (Hanes, et al., Psychopharmacol. Bull., 24, 62 (1988));

Risperidone, 3-[2-[4-(6-fluoro-1,2-benzisoxazol-3-yl)piperidino]ethyl]-2-methyl-6,7,8,9-tetrahydro-4H-pyrido-[1,2-a]pyrimidin-4-one, and its use in the treatment of psychotic diseases are described in U.S. Pat. No. 4,804,663, which is herein incorporated by reference in its entirety;

Sertindole, 1-[2-[4-[5-chloro-1-(4-fluorophenyl)-1H-indol-3-yl]-1-piperidinyl]ethyl]imidazolidin-2-one, is described in U.S. Pat. No. 4,710,500. Its use in the treatment of schizophrenia is described in U.S. Pat. Nos. 5,112,838 and 5,238,945. U.S. Pat. Nos. 4,710,500; 5,112,838; and 5,238,945 are herein incorporated by reference in their entirety;

Quetiapine, 5-[2-(4-dibenzo[b,f][1,4]thiazepin-11-yl-1-piperazinyl) ethoxy]ethanol, and its activity in assays which demonstrate utility in the treatment of schizophrenia are described in U.S. Pat. No. 4,879,288, which is herein incorporated by reference in its entirety. Quetiapine is typically administered as its (E)-2-butenedioate (2:1) salt;

Aripiprazole, 7-{4-[4-(2,3-dichlorophenyl)-1-piperazinyl]-butoxy}-3,4-dihydro carbostyril or 7-{4-[4-(2,3-dichlorophenyl)-1-piperazinyl]-butoxy}-3,4-dihydro-2(1H)-quinolinone, is an atypical antipsychotic agent used for the treatment of schizophrenia and described in U.S. Pat. No. 4,734,416 and U.S. Pat. No. 5,006,528, which are herein incorporated by reference in their entirety;

Amisulpride is described in U.S. Pat. No. 4,401,822;

Asenapine, trans-5-chloro-2-methyl-2,3,3a, 12b-tetrahydro-1H-dibenz[2,3:6,7]oxepino[4,5-c]pyrrole. Preparation and use of asenapine is described in U.S. Pat. Nos. 4,145,434 and 5,763,476, which are incorporated herein in their entireties by reference.

A preferred combination is ziprasidone with a GABA modulator. The term “GABA”, where used in the description and the appendant claims, is synonymous with the term “gamma-aminobutyric acid.” These terms are used interchangeably throughout the description and appendant claims.

The term “GABA modulator” as used herein refers to a compound that either is structurally related to the neurotransmitter GABA but does not interact with the GABA receptor (e.g. gabapentin), or interacts with the GABA receptors, or is converted metabolically into GABA or a GABA agonist; or is an inhibitor of GABA uptake or degradation; or is a GABA receptor subtype-selective antagonist and/or agonist. This definition includes pharmaceutically acceptable salts, prodrugs or pharmaceutically acceptable salts of said prodrugs.

The GABA modulators suitable for use herein include, but are not limited to, muscimol, progabide, riluzole, baclofen, gabapentin (Neurontin®), vigabatrin, tiagabine (Gabitril®), lamotrigine (Lamictal®), pregabalin, topiramate (Topamax®), a prodrug thereof or a pharmaceutically acceptable salt of the GABA modulator or prodrug thereof. It will be recognized by those skilled in the art in light of this disclosure that other GABA agonists are also useful in the combinations, pharmaceutical compositions, methods and kits of this invention.

The GABA modulators disclosed herein are prepared by methods well known to those skilled in the art. Specifically, the following patents and patent applications, each of which is hereby incorporated herein by reference, exemplify GABA modulators which can be used in the combinations, pharmaceutical compositions, methods and kits of this invention, and refer to methods of preparing those GABA modulators: U.S. Pat. No. 3,242,190 (specifically, muscimol); U.S. Pat. No. 4,094,992 (specifically, progabide); U.S. Pat. No. 4,370,338 (specifically, riluzole); U.S. Pat. No. 3,471,548 (specifically, baclofen); U.S. Pat. No. 4,024,175 (specifically, gabapentin); U.S. Pat. No. 3,960,927 (specifically, vigabatrin); U.S. Pat. No. 5,010,090 (specifically, tiagabine); U.S. Pat. No. 4,602,017 (specifically, lamotrigine); U.S. Pat. No. 6,028,214 (specifically, pregabalin); and U.S. Pat. No. 4,513,006 (specifically, topiramate).

Gabapentin, 1-(aminomethyl)cyclohexane acetic acid, is an anticonvulsant indicated as adjunctive therapy in the treatment of partial seizures with and without secondary generalization in adults with epilepsy. Gabapentin and its methods of use is described in U.S. Pat. Nos. 4,024,175 and 4,087,544 incorporated herein by reference in their entirety.

It will be recognized that certain of the GABA modulators used in the pharmaceutical compositions, methods and kits of this invention contain either a free carboxylic acid or a free amine group as part of the chemical structure. Thus, this invention includes pharmaceutically acceptable salts of those carboxylic acids or amine groups.

For use in medicine, pharmaceutically acceptable salts may be useful in the preparation of the compounds according to the invention. Suitable pharmaceutically acceptable salts of the compounds of this invention include acid addition salts which may, for example, be formed by mixing a solution of the compound according to the invention with a solution of a pharmaceutically acceptable acid such as hydrochloric acid, sulfuric acid, methanesulphonic acid, fumaric acid, maleic acid, succinic acid, acetic acid, benzoic acid, oxalic acid, citric acid, tartaric acid, carbonic acid or phosphoric acid. Furthermore, where the compounds of the invention carry an acidic moiety, suitable pharmaceutically acceptable salts thereof may include alkali metal salts, e.g. sodium or potassium salts; alkaline earth metal salts, e.g. calcium or magnesium salts; and salts formed with suitable organic ligands, e.g. quaternary ammonium salts.

Where the GABA modulators of use in the invention have at least one asymmetric center, they may accordingly exist as enantiomers. Where the compounds according to the invention possess two or more asymmetric centers, they may additionally exist as diastereoisomers. It is to be understood that all such isomers and mixtures thereof in any proportion are encompassed within the scope of the present invention. Gabapentin may be in the form of the crystalline monohydrate as described in EP340677 which is incorporated herein by reference or the anhydrous crystalline form as described in WO 03031391.

The expression “pharmaceutically acceptable salts” includes both pharmaceutically acceptable acid addition salts and pharmaceutically acceptable cationic salts. The expression “pharmaceutically-acceptable cationic salts” is intended to define but is not limited to such salts as the alkali metal salts, (e.g., sodium and potassium), alkaline earth metal salts (e.g., calcium and magnesium), aluminum salts, ammonium salts, and salts with organic amines such as benzathine (N,N′-dibenzylethylenediamine), choline, diethanolamine, ethylenediamine, meglumine (N-methylglucamine), benethamine (N-benzylphenethylamine), diethylamine, piperazine, tromethamine (2-amino-2-hydroxymethyl-1,3-propanediol) and procaine. The expression “pharmaceutically-acceptable acid addition salts” is intended to define but is not limited to such salts as the hydrochloride, hydrobromide, sulfate, hydrogen sulfate, phosphate, hydrogen phosphate, dihydrogenphosphate, acetate, succinate, citrate, methanesulfonate (mesylate) and p-toluenesulfonate (tosylate) salts.

The pharmaceutically-acceptable cationic salts of GABA modulators containing free carboxylic acids may be readily prepared by reacting the free acid form of the GABA modulator with an appropriate base, usually one equivalent, in a co-solvent. Typical bases are sodium hydroxide, sodium methoxide, sodium ethoxide, sodium hydride, potassium methoxide, magnesium hydroxide, calcium hydroxide, benzathine, choline, diethanolamine, piperazine and tromethamine. The salt is isolated by concentration to dryness or by addition of a non-solvent. In many cases, salts are preferably prepared by mixing a solution of the acid with a solution of a different salt of the cation (e.g., sodium or potassium ethylhexanoate, magnesium oleate), employing a solvent (e.g., ethyl acetate) from which the desired cationic salt precipitates, or can be otherwise isolated by concentration and/or addition of a non-solvent.

The pharmaceutically acceptable acid addition salts of GABA modulators containing free amine groups may be readily prepared by reacting the free base form of the GABA modulator with the appropriate acid. When the salt is of a monobasic acid (e.g., the hydrochloride, the hydrobromide, the p-toluenesulfonate, the acetate), the hydrogen form of a dibasic acid (e.g., the hydrogen sulfate, the succinate) or the dihydrogen form of a tribasic acid (e.g., the dihydrogen phosphate, the citrate), at least one molar equivalent and usually a molar excess of the acid is employed. However, when such salts as the sulfate, the hemisuccinate, the hydrogen phosphate or the phosphate are desired, the appropriate and exact chemical equivalents of acid will generally be used. The free base and the acid are usually combined in a co-solvent from which the desired salt precipitates, or can be otherwise isolated by concentration and/or addition of a non-solvent.

The anticonvulsants disclosed herein are prepared by methods well known to those skilled in the art. Specifically, the following patents and patent applications, each of which is hereby incorporated herein by reference, exemplify anticonvulsants which can be used in the combinations, pharmaceutical compositions, methods and kits of this invention, and refer to methods of preparing those anticonvulsants:

Anticonvulsants contemplated as the second component include, but are not limited to, phenyloin, carbamezepine, valproic acid, lamotrigine and topiramate;

Carbamezepine, 5H-dibenz [b,f]azepine-5-carboxamide is an anticonvulsant and analgesic marketed for trigeminal neuralgia; U.S. Pat. No. 2,948,718 (herein incorporated herein by reference in its entirety), discloses carbamezepine and methods of use;

Phenyloin, 5,5-diphenyl-2,4-imidazolidinedione, is a well-known anticonvulsant; U.S. Pat. No. 2,409,654 discloses phenyloin and methods of use; incorporated herein by reference in its entirety.

Valproic Acid, 2-propylpentanoic acid or dispropylacetic acid is a well known antiepileptic agent which dissociates to the valproate ion in the gastrointestinal tract; various pharmaceutically acceptable salts are disclosed in U.S. Pat. No. 4,699,927; Valproic acid is prepared as disclosed in Carraz et al., Therapie, 1965, 20, 419) incorporated herein by reference in its entirety;

Lamotrigine, 6-(2,3-dichlorophenyl)-1,2,4-trizine-3,5-diamine is an antiepileptic drug indicated as adjunctive therapy in the treatment of partial seizures in adults with epilepsy. Lamotrigine and its uses is disclosed in U.S. Pat. No. 4,486,354, incorporated herein by reference in its entirety; and

Topiramate, 2,3:4,5-di-O-(1-isopropylidine)-3-D-fructopyranose sulphamate is an antiepileptic indicated for the treatment of refractory partial seizures, with or without secondary generalization and disclosed in U.S. Pat. No. 4,513,006 incorporated herein by reference in its entirety.

The benzodiazepines are used as antianxiety agents and in psychiatric disorders in which anxiety is a prominent feature. For example, combination treatment with a benzodiazepine plus a typical antipsychotic (often haloperidol IM 5-10 mg plus lorazepam 1-2 mg) is commonly employed. However, this combination may be associated with intolerable side effects, particularly acute dystonia with conventional antipsychotics and excessive sedation with benzodiazepines. Also, some clinicians avoid benzodiazepines in agitation associated with intoxication.

Benzodiazepines are also associated with excessive sedation, confusion, disinhibition, ataxia, nausea and vomiting, respiratory depression, asymptomatic tachypnea, and tachycardia (J. Modell, J Clin Psychopharmacol. 6:385-387, 1986). According to the invention it has surprisingly been found that an atypical antipsychotic counteracts the typical sedative and hypnotic effects of benzodiazepines.

Thus, by administering, in accordance with the principle of the present invention, an atypical antipsychotic such as ziprasidone to patients treated with benzodiazepines, it will be possible, because of the counteraction of the sedative and hypnotic effects, to use effective dosages of the benzodiazepines even where high dosages are necessary to obtain an effect, without disabling the patients from living a normal daily life.

In the present context, the term “a benzodiazepine” or “benzodiazepines” designate benzodiazepine as well as derivatives thereof which are normally classified as benzodiazepines in pharmaceutical textbooks such as, e.g., Ernst Mutschler, Arzneimittelwirkungen, Lehrbuch der Pharmaceutical makologie and Toxikologie, Aug. 5, 1986, Wissenschaftliche Verlagsgasellschaft mbk, Stuttgart, including, e.g., diazepam, dipotassium chlorazepate, chlorasepate, chlordiazapide, medazepam, flurazepam, clobasam, clonazepam, nitrasepam, flunitrasepam, astazolam, bromazepam, alprazolam, lorazepam, lormetasepam, oxazepam, temazepam, brotizolam, triazolam, chlordiazepam, halazepam, or prazepam. As defined herein the term benzodiazepines also refers to benzodiazepine receptor subtype compounds as well as pharmaceutically acceptable salts of benzodiazepines, prodrugs of benzodiazepines and pharmaceutically acceptable salts of benzodiazepine prodrugs.

Some benzodiazepines are used for their sedative or hypnotic effect; these benzodiazepines are typically those having a short half life. Other benzodiazepines are used for other effects where the sedative or the hypnotic effects are considered undesirable or even side effects of the benzodiazepine. These benzodiazepines are, e.g., diazepam, dipotassium chlorazepate, chlorazepate, chlordiazepide, medazepam, clobazam, clonazepam, estazolam, bromasepam, alprazolam, lorazepam, lormetazepam, oxazepam, brotizolam, chlordiazepam, halazepam, or prazepam.

The diseases treated with benzodiazepines constitute a broad spectrum of diseases because of the many effects of the benzodiazepines. Diseases where the sedative or hypnotic effects of the benzodiazepines are undesirable are diseases in connection with which the principle of the present invention is particularly important. Especially the treatment of the following diseases is accomplished by the drug combinations of the present invention: treatment-resistant anxiety, psychotic disorders or conditions, psychotic symptoms. These diseases may benefit from the use of both a benzodiazepine and an antypical antipsychotic in accordance with the principle of the invention, as these diseases are known to require high dosages of benzodiazepine in order to obtain the benefit of the benzodiazepine therapy. However, the high dosages, on the other hand, incur the above-mentioned severe disadvantages due to the sedative and hypnotic effects if no administration of the atypical antipsychotic is performed in connection with the benzodiazepine treatment.

Psychotic disorders or conditions, such as schizophrenia, schizoaffective disorder, schizophreniform disorder, and schizotypical disorder are conditions in which benzodiazepine therapy, such as treatment with clonazepam, is important. According to the present invention, these conditions can now also be treated with an atypical antipsychotic in combination with a benzodiazepine.

The atypical antipsychotic can be administered simultaneously with the benzodiazepine, either as separate dosage forms in a kit product, or as one combined dosage form containing both the atypical antipsychotic and the benzodiazepine.

The effects of a pharmaceutical composition comprising ziprasidone and a GABA modulator, or ziprasidone and a benzodiazepine of the present invention can be examined by using one or more of the published models of anxiety well known in the art. The effects of a pharmaceutical composition comprising ziprasidone and a benzodiazepine, or ziprasidone and an anticonvulsant of the present invention can be examined by using one or more of the published models of psychotic disorders or conditions well known in the art. The effects of a pharmaceutical composition comprising ziprasidone and an anticonvulsant of the present invention can be examined by using one or more of the published models of mood disorders such as bipolar disorder which are well known in the art.

The pharmaceutical compositions containing ziprasidone and a GABA modulator or ziprazidone and a benzodiazepine of the present invention are particularly useful for the prevention of, reducing the development of, or reversal of, treatment-resistant anxiety disorders and are therefore particularly useful in the treatment of obsessive-compulsive disorder or post-traumatic stress disorder. This effect can be demonstrated, for example, by measuring markers such the Clinician Administered PTSD Scale or the Eysenck Personality Inventory and has been shown in clinical studies (MI Butterfield et al, 16 Int'l Clin Psychopharmacol 197-203, 2001).

The pharmaceutical compositions containing ziprasidone and an anticonvulsant or ziprazidone and a benzodiazepine of the present invention are particularly useful for the prevention of, reducing the development of, or reversal of, psychotic disorders, conditions or symptoms and are therefore particularly useful in the treatment of schizophrenia, schizophreniform disorder, schizoaffective disorder or delusional disorder. This can be demonstrated, for example, by measuring markers such Positive or Negative Syndrome Scale (PANSS) and Scales for the Assessment of Negative Symptoms (SANS) or BPRS scores (Kay et al, Schizophrenia Bulletin 13:261-276, 1987), or in various animal models such as PCP or methamphetamine induced locomotor test or the conditioned avoidance response test.

The pharmaceutical compositions containing ziprasidone and an anticonvulsant are particularly useful for the prevention of, reducing the development of, or reversal of, mood disorders and are therefore particularly useful in the treatment of bipolar disorder, bipolar depression or unipolar depression. This can be demonstrated, for example, by measuring the symptomatic picture and using various animal models such as the “mouse behavioral despair test.”

In general, ziprasidone employed in the combinations, pharmaceutical compositions, methods and kits of this invention, will be administered at dosages between about 20 and about 460 mg per day, preferably from about 40 mg to about 200 mg, and most preferably 40 mg to 160 mg together with therapeutically effective amounts of the second therapeutic agent in single or divided doses.

The term “therapeutically effective amount” as used herein refers to a sufficient amount of the compound to treat treatment-resistant anxiety disorders, mood disorders and psychotic disorders or conditions at a reasonable benefit/risk ratio applicable to any medical treatment.

The specific therapeutically effective dose level for any particular patient will depend upon a variety of factors including the disorder being treated and the severity of the disorder; activity of the specific compound employed; the specific composition employed; the age. However, some variation in dosage will necessarily occur depending upon the condition of the subject being treated. The person responsible for administration will, in any event, determine the appropriate dose for the individual subject.

The following dosage amounts and other dosage amounts set forth elsewhere in this description and in the appendant claims are for an average human subject having a weight of about 65 kg to about 70 kg. The skilled practitioner will readily be able to determine the dosage amount required for a subject whose weight falls outside the 65 kg to 70 kg range, based upon the medical history of the subject. All doses set forth herein, and in the appendant claims, are daily doses.

In general, in accordance with this invention, the above GABA modulators used in the combinations, pharmaceutical compositions, methods and kits of this invention will be administered in a dosage amount of about 4 mg/kg body weight of the subject to be treated per day to about 60 mg/kg body weight of the subject to be treated per day, in single or divided doses. However, some variation in dosage will necessarily occur depending upon the condition, age as well as factors which may alter pharmacokinetics of absorption, distribution, metabolism and excretion in the subject being treated. The person responsible for administration will, in any event, determine the appropriate dose for the individual subject. In particular, when used as the GABA modulator in this invention, pregabalin will be dosed at about 100 mg to about 1500 mg per day; and preferably about 300 mg to about 1200 mg per day; gabapentin will be dosed at about 100 mg to about 4000 mg per day, and preferably about 600 mg to about 3600 mg per day.

In general, in accordance with this invention, the above anticonvulsants used in the combinations, pharmaceutical compositions, methods and kits of this invention will be administered in a dosage amount of about 1 mg/kg body weight of the subject to be treated per day to about 10 mg/kg body weight of the subject to be treated per day, in single or divided doses. However, some variation in dosage will necessarily occur depending upon the condition of the subject being treated. The person responsible for administration will, in any event, determine the appropriate dose for the individual patient. In particular, when used as the anticonvulsant in this invention, phenyloin will be dosed at about 10 mg to about 1500 mg per day and preferably about 50 mg to about 1200 mg per day or doses to achieve serum levels in the range of about 10-20 mcg/mL; valproic acid will be dosed at about 1 mg/kg/day to about 100 mg/kg/day, and preferably about 5 mg/kg/day to about 70 mg/kg/day.

In general, in accordance with this invention, the above benzodiazepines used in the combinations, pharmaceutical compositions, methods and kits of this invention will be administered in a dosage amount of about 0.001 mg to about 200 mg, in single or divided doses. However, some variation in dosage will necessarily occur depending upon the condition, age and pharmacokinetic altering physiology of the subject being treated. The person responsible for administration will, in any event, determine the appropriate dose for the individual patient. In particular, when used as the benzodiazepine in this invention, diazepam will be dosed at about 1 mg to about 40 mg per day; clonazepam will be dosed at about 0.001 mg/kg/day to about 1 mg/kg/day, and more preferably at about 0.01 mg/kg/day to about 0.2 mg/kg/day.

The exact formulation, route of administration, and dosage can be chosen by the individual physician in view of the patient's condition. Dosage amount and interval can be adjusted individually to provide plasma levels of the active moiety which are sufficient to maintain therapeutic effects

It will be recognized by a skilled person that the free base form or other salt forms of the above GABA modulators, anticonvulsants and benzodiazepines may be used in this invention. Calculation of the dosage amount for these other forms of the free base form or other salt forms of a particular GABA modulator, anticonvulsant or benzodiazepine is easily accomplished by performing a simple ratio relative to the molecular weights of the species involved.

The products of the present invention are of use in the treatment and/or prevention of a variety of disorders of the central nervous system. Such disorders include treatment-resistant anxiety disorders, such as obsessive-compulsive disorder, stress disorders including post-traumatic and acute stress disorder, and generalized or substance-induced anxiety disorder; neuroses; depressive or bipolar disorders, for example single-episode or recurrent major depressive disorder, dysthymic disorder, bipolar I and bipolar II manic disorders, and cyclothymic disorder.

The products of the present invention have the advantage that they surprisingly provide relief from anxiety more rapidly than would be expected from administration of either compound alone. They are useful in reducing the complications associated with treatment-resistant anxiety disorders, including premature mortality and suicide.

The term “treatment-resistant”, as in “a method of treating a disorder”, refers to reversing, alleviating, or inhibiting the progress of the disorder to which such term applies, or one or more symptoms of the disorder. For example, in some clinical studies it is defined as patients with a principal DSM-IV diagnosis of generalized anxiety disorder who have not responded sufficiently after an adequate trial (4-8 weeks) of first-line anti-anxiety agents such as SSRIs, buspirone or a benzodiazepine. As used herein, the term also encompasses, depending on the condition of the patient, preventing the disorder, including preventing onset of the disorder or of any symptoms associated therewith, as well as reducing the severity of the disorder or any of its symptoms prior to onset, or to preventing a recurrence of a disorder.

Examples of treatment-resistant anxiety disorders that can be treated according to the present invention include, but are not limited to, treatment-resistant obsessive-compulsive disorder, treatment-resistant posttraumatic stress disorder, generalized or substance-induced anxiety disorder; neuroses and panic disorder.

The meanings attributed to the different types and subtypes of anxiety disorders are as stated in DSM-IV-TR the contents of which are incorporated by reference herein. (Diagnostic and Statistical Manual of Mental Disorders”, 4^{th} ed, American Psychiatric Assoc., Washington, D.C., 2002, p. 429-484). Though classified as an anxiety disorder in DSM-IV, PTSD is accompanied by psychotic symptoms in almost half of patients.

Examples of psychotic disorders that can be treated according to the present invention include, but are not limited to, schizophrenia, for example of the paranoid, disorganized, catatonic, undifferentiated, or residual type; schizophreniform disorder; schizoaffective disorder, for example of the delusional type or the depressive type; delusional disorder; brief psychotic disorder; shared psychotic disorder; psychotic disorder due to a general medical condition; substance-induced psychotic disorder, for example psychosis induced by alcohol, amphetamine, cannabis, cocaine, hallucinogens, inhalants, opioids, or phencyclidine; personality disorder of the paranoid type; personality disorder of the schizoid type; psychotic disorder not otherwise specified.

The meanings attributed to the different types and subtypes of psychotic disorders are as stated in DSM-IV-TR the contents of which are incorporated by reference herein. (Diagnostic and Statistical Manual of Mental Disorders”, 4^{th} ed, American Psychiatric Assoc., Washington, D.C., 2002, p. 297-343).

Schizophrenia as used herein refers to a disorder that lasts for at least 6 months and includes at least one month of active-phase symptoms (i.e., two [or more] of the following: delusions, hallucinations, disorganized speech, grossly disorganized or catatonic behavior, negative symptoms) (Diagnostic and Statistical Manual of Mental Disorders, DSM-IV-TR, 4^{th} ed, American Psychiatric Assoc., Washington, D.C., 2002).

Schizoaffective disorder is defined as a disorder in which a mood episode and the active-phase symptoms of schizophrenia occur together and were preceded or are followed by at least 2 weeks of delusions or hallucinations without prominent mood symptoms (Diagnostic and Statistical Manual of Mental Disorders, DSM-IV-TR, 4^{th} ed, American Psychiatric Assoc., Washington, D.C., 2002).

Schizophreniform disorder is defined as a disorder characterized by a symptomatic presentation that is equivalent to schizophrenia except for its duration (i.e., the disturbance lasts from 1 to 6 months) and the absence of a requirement that there be a decline in functioning (Diagnostic and Statistical Manual of Mental Disorders, DSM-IV-TR, 4^{th} ed, American Psychiatric Assoc., Washington, D.C., 2002).

Schizotypical disorder is defined as a lifetime pattern of social and interpersonal deficits characterized by an inability to form close interpersonal relationships, eccentric behavior, and mild perceptual distortions.

The combinations of ziprasidone with anticonvulsant drugs or ziprasidone and benzodiazepines in the present invention can be used to treat other psychotic disorders such as delusional disorder; brief psychotic disorder; shared psychotic disorder; substance-induced psychotic disorder, for example psychosis induced by alcohol, amphetamine, cannabis, cocaine, hallucinogens, inhalants, opioids, or phencyclidine; psychotic disorder due to a general medical condition; personality disorder of the paranoid type; personality disorder of the schizoid type; and psychotic disorder not otherwise specified.

For example, “treating schizophrenia, or schizophreniform or schizoaffective disorder” as used herein also encompasses treating one or more symptoms (positive, negative, and other associated features) of said disorders, for example treating, delusions and/or hallucination associated therewith. Other examples of symptoms of schizophrenia and schizophreniform and schizoaffecctive disorders include disorganized speech, affective flattening, alogia, anhedonia, inappropriate affect, dysphoric mood (in the form of, for example, depression, anxiety or anger), and some indications of cognitive dysfunction.

Delusional disorder as referred to herein is characterized by at least 1 month of nonbizarre delusions without other active-phase symptoms of schizophrenia. (Diagnostic and Statistical Manual of Mental Disorders, DSM-IV-TR, 4^{th} ed, American Psychiatric Assoc., Washington, D.C., 2002).

Brief psychotic disorder is a disorder that lasts more than 1 day and remits by 1 month. (Diagnostic and Statistical Manual of Mental Disorders, DSM-IV-TR, 4^{th} ed, American Psychiatric Assoc., Washington, D.C., 2002).

Shared psychotic disorder is characterized by the presence of a delusion in an individual who is influenced by someone else who has a longer-standing delusion with similar content. (Diagnostic and Statistical Manual of Mental Disorders, DSM-IV-TR, 4^{th} ed, American Psychiatric Assoc., Washington, D.C., 2002).

Psychotic disorder due to a general medical condition is characterized by psychotic symptoms judged to be a direct physiological consequence of a general medical condition. (Diagnostic and Statistical Manual of Mental Disorders, DSM-IV-TR, 4^{th} ed, American Psychiatric Assoc., Washington, D.C., 2002).

Psychotic disorder not otherwise specified is a psychotic presentation that does not meet the criteria for any of the specific psychotic disorders defined in the DSM-IV-TR (American Psychiatric Assoc., Washington, D.C., 2002).

In another embodiment, the compounds used in the present invention are useful to treat other disorders that may present with psychotic symptoms as associated features such as dementia of the Alzheimer's type; substance-induced delirium; and major depressive disorder with psychotic features.

In a preferred embodiment, the compounds used in the present invention are useful for treating schizophrenia, a schizoaffective disorder, schizophreniform disorder, or a schizotypical disorder.

The combinations of ziprasidone and an anticonvulsant may be used to treat mood disorders, formerly designated as “affective disorders.” Although mood disorders are not a clearly delineated group of illnesses they include unipolar and bipolar depression, generalized anxiety disorder, and more specific anxiety disorders such as agoraphobia, panic disorder and social phobia, obsessive-compulsive disorder and post traumatic stress disorder (PTSD). There is a high level of similarity and co-morbidity between these illnesses and clinicians may consider them as a single group.

The meanings attributed to the different types and subtypes of mood disorders are as stated in DSM-IV-TR under depressive disorders (“unipolalar depression”) and bipolar disorders, generalized anxiety disorder, and more specific anxiety disorders such as agoraphobia, panic disorder and social phobia, obsessive-compulsive disorder and post traumatic stress disorder (PTSD), the contents of which are incorporated by reference herein. (Diagnostic and Statistical Manual of Mental Disorders”, 4^{th} ed, American Psychiatric Assoc., Washington, D.C., 2002, p. 345-484).

The term “affective disorder” as used herein is interchangeable with the term “mood disorders” and refers to disorders that are characterized by changes in mood as the primary clinical manifestation, for example, depression.

The expression “prodrug” refers to compounds that are drug precursors which, following administration, release the drug in vivo via a chemical or physiological process (e.g., a prodrug on being brought to the physiological pH or through enzyme action is converted to the desired drug form).

The present invention includes within its scope the use of prodrugs of ziprasidone, GABA modulators, benzodiazepines or anticonvulsant drugs. In general, such prodrugs will be functional derivatives of these compounds which are readily convertible in vivo. Conventional procedures for the selection and preparation of suitable prodrug derivatives are described, for example, in Design of Prodrugs, ed. H. Bundgaard, Elsevier, 1985 and can be achieved using methods well known to those skilled in the art. All such prodrugs are within the scope of the combinations, pharmaceutical compositions, methods and kits of this invention.

The chemist of ordinary skill in the art will also recognize that certain compounds within the scope of this invention can exist in zwitterionic form, i.e., that certain compounds contain an amine portion and a carboxylic acid portion, which, depending upon the pH of the solution, may exist as a free amine and a free carboxylic acid or as a zwitterion in which the amine is protonated to form an ammonium ion and the carboxylic acid is deprotonated to form a carboxylate ion. All such zwitterions are included in this invention.

The chemist of ordinary skill in the art will also recognize that the pharmaceutical combinations contemplated by the present invention can exist in different stereoisomers. Specific stereoisomers may exhibit an ability to treat mental disorders with a more favorable efficacy or safety profile. The present invention includes all possible stereoisomers and geometric isomers of the active ingredients of each pharmaceutical combination, and includes not only racemic compounds but also optical isomers as well. In situations where tautomers, i.e. that an equilibrium exists between two isomers which are in rapid equilibrium with each other are possible, the present invention is intended to include all tautomeric forms.

The combinations of the present invention can be administered in a standard manner for the treatment of treatment-resistant anxiety disorders, psychotic disorders, or mood disorders such as orally, parenterally, transmucosally (e.g., sublingually or via buccal administration), topically, transdermally, rectally, via inhalation (e.g., nasal or deep lung inhalation). Parenteral administration includes, but is not limited to intravenous, intraarterial, intraperitoneal, subcutaneous, intramuscular, intrathecal, and intraarticular, or via a high pressure technique, like Powderject.™

For buccal administration, the composition can be in the form of tablets or lozenges formulated in conventional manner. For example, tablets and capsules for oral administration can contain conventional excipients such as binding agents (for example, syrup, acacia, gelatin, sorbitol, tragacanth, mucilage of starch or polyvinylpyrrolidone), fillers (for example, lactose, sugar, microcrystalline cellulose, maize-starch, calcium phosphate or sorbitol), lubricants (for example, magnesium stearate, stearic acid, talc, polyethylene glycol or silica), disintegrants (for example, potato starch or sodium starch glycollate), or welting agents (for example, sodium lauryl sulfate). The tablets can be coated according to methods well known in the art.

Such preparations can also be formulated as suppositories, e.g., containing conventional suppository bases, such as cocoa butter or other glycerides. Compositions for inhalation typically can be provided in the form of a solution, suspension, or emulsion that can be administered as a dry powder or in the form of an aerosol using a conventional propellant, such as dichlorodifluoromethane or trichlorofluoromethane. Typical topical and transdermal formulations comprise conventional aqueous or nonaqueous vehicles, such as eye drops, creams, ointments, lotions, and pastes, or are in the form of a medicated plaster, patch, or membrane.

Additionally, compositions of the present invention can be formulated for parenteral administration by injection or continuous infusion. Formulations for injection can be in the form of suspensions, solutions, or emulsions in oily or aqueous vehicles, and can contain formulation agents, such as suspending, stabilizing, and/or dispersing agents. Alternatively, the active ingredient can be in powder form for constitution with a suitable vehicle (e.g., sterile, pyrogen-free water) before use.

A composition in accordance with the present invention also can be formulated as a depot preparation. Such long acting formulations can be administered by implantation (for example, subcutaneously or intramuscularly) or by intramuscular injection. Accordingly, the compounds of the invention can be formulated with suitable polymeric or hydrophobic materials (e.g., an emulsion in an acceptable oil), ion exchange resins, or as sparingly soluble derivatives (e.g., a sparingly soluble salt).

Solubilized forms of aryl-heterocyclics such as zirpasidone, pharmaceutically acceptable salts there, or prodrugs thereof, or pharmaceutically acceptable salts of prodrugs thereof, associated with (or at levels even greater than) immediate release can be fabricated into depot formulations. For example, a pharmaceutical kit comprising ziprazidone, ziprasidone salts or prodrugs thereof, or pharmaceutically acceptable salts of ziprasidone prodrugs, which can be solubilized or unsolubilized; and a constituting liquid vehicle comprised of a viscosity agent with the proviso that when the ziprasidone compound is unsolubilized, the aqueous liquid further comprises a solubilizer.

Ziprazidone depot formulation in the form of a suspension are described in U.S. Patent Application Ser. No. 60/42195, filed Oct. 25, 2002 and incorporated herein by reference in its entirety. Novel injectable depot formulations of ziprasidone are described in U.S. Patent Application Ser. No. 60/421,473, filed Oct. 25, 2002 and incorporated herein by reference in its entirety.

For oral administration a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols.

Alternatively, the compounds of the present invention can be incorporated into oral liquid preparations such as aqueous or oily suspensions, solutions, emulsions, syrups, or elixirs, for example. Moreover, formulations containing these compounds can be presented as a dry product for constitution with water or other suitable vehicle before use. Such liquid preparations can contain conventional additives, such as suspending agents, such as sorbitol syrup, synthetic and natural gums such as tragacanth, acacia, alginate, dextran, sodium carboxymethylcellulose, methylcellulose, polyvinyl-pyrrolidone or gelatin, glucose/sugar syrup, gelatin, hydroxyethylcellulose, hydroxypropylmethylcellulose, aluminum stearate gel, emulsifying agents, such as lecithin, sorbitan monooleate, or acacia; nonaqueous vehicles (which can include edible oils), such as almond oil, fractionated coconut oil, oily esters, propylene glycol, and ethyl alcohol; and preservatives, such as methyl or propyl p-hydroxybenzoate and sorbic acid. The liquid forms in which the compositions of the present invention may be incorporated for administration orally or by injection include aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles.

When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatin.

The combinations of this invention can also be administered in a controlled release formulation such as a slow release or a fast release formulation. Such controlled release formulations of the combinations of this invention may be prepared using methods well known to those skilled in the art. The method of administration will be determined by the attendant physician or other person skilled in the art after an evaluation of the patient's condition and requirements.

The pharmaceutical compositions of the present invention can consist of a combination of immediate release and controlled release characteristics. Such compositions can take the form of combinations of the active ingredients that range in size from nanoparticles to microparticles or in the form of a plurality of pellets with different release rates. The tablet or capsule composition of the present invention can contain an atypical antipsychotic in sustained or controlled release form and, a second therapeutic agent in an immediate release form. Alternatively, the atypical antipsychotic can be in immediate release form and the second therapeutic agent can be in sustained or controlled release form.

The combinations of this invention can also be administered in parenteral form. For parenteral administration, solutions in sesame or peanut oil or in aqueous propylene glycol can be employed, as well as sterile aqueous solutions of the corresponding water-soluble salts. Such aqueous solutions can be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

Methods of preparing various pharmaceutical compositions with a certain amount of active ingredient are known, or will be apparent in light of this disclosure, to those skilled in this art. For examples, methods of preparing pellets are described in Remington: The Science and Practice of Pharmacy, Mack Publishing Company, Easton, Pa., 19th Edition (1995). Prolonged release pellets are prepared by either coating immediate release pellets or via matrix systems. Coating may be carried out, for example, in coating pans or in fluid bed coater-driers. Extrusion and subsequent spheronization is a long-known method for the preparation of pharmaceutical pellets (J. W. Conine et al., Drug & Cosmetic Ind. 106, 38-41 (1970)). However, other methods such as pelletization may be utilized. Particles may be agglomerated to form spherical granules or pellets, in a high speed mixer granulator, or rotary fluid bed agglomerator. These methods are described by K. W. Olson and A. M. Mehta, Int.J.Pharm.Tech&.Prod.Mfr. 6 18-24, 1985. Pellets may be also prepared by extrusion of wet masses or melts followed by spheronisation, for example as described in C. Vervaet, L. Baert & J. P. Remon Int.J.Pharm. 116 (1995) 131-146. Excipients used are typically those with plastic qualities such as microcrystalline cellulose, but also mannitol. Small quantities of a polymeric binder are generally added. Surfactants such as sodium dodecyl sulphate may also be incorporated to give easier extrusion.

Pharmaceutical compositions according to the invention can contain 0.1%-95% of the therapeutic agents of this invention, preferably 1%-70%. In any event, the composition or formulation to be administered will contain a quantity of therapeutic agent(s) according to the invention in an amount effective to treat the condition or disease of the subject being treated.

The two different compounds of this invention can be co-administered simultaneously or sequentially in any order, or as a single pharmaceutical composition comprising, for example, ziprasidone and a GABA modulator, or ziprasidone and an anticonvulsant, or ziprasidone and a benzodiazepine as described above.

Since the present invention has an aspect that relates to the treatment of the disease/conditions described herein with a combination of active ingredients which can be administered separately, the invention also relates to combining separate pharmaceutical compositions in kit form. The kit comprises two separate pharmaceutical compositions: ziprasidone and a GABA modulator, a prodrug thereof or a pharmaceutically acceptable salt of said GABA modulator or prodrug; or ziprasidone and an anticonvulsant, a prodrug thereof or a pharmaceutically acceptable salt of said GABA modulator or prodrug; ziprasidone and a benzodiazepine, a prodrug thereof or a pharmaceutically acceptable salt of said GABA modulator or prodrug. The kit includes a container for containing the separate compositions such as a divided bottle or a divided foil packet. Typically the kit includes directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc . . . Second Week, Monday, Tuesday, . . . ” etc. Other variations of memory aids will be readily apparent to the skilled practitioner. A “daily dose” can be a single tablet or capsule or several pills or capsules to be taken on a given day. Also, a daily dose of the ziprasidone can consist of one tablet or capsule while a daily dose of the anticonvulsant, benzodiazepine or GABA modulator can consist of several tablets or capsules or vice versa. The memory aid should reflect this.

In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

In another embodiment of the present invention, the treatment of treatment-resistant anxiety disorder in a patient the method of the present invention can include administering a triple combination pharmaceutical composition containing an amount of a first therapeutic agent, said first therapeutic agent being ziprasidone;
an amount of a second therapeutic agent, said second therapeutic agent being a GABA modulator, a prodrug thereof or a pharmaceutically acceptable salt of said GABA modulator or said prodrug; and
an amount of a third therapeutic agent, said third therapeutic agent being a benzodiazepine, a prodrug thereof or a pharmaceutically acceptable salt of said benzodiazepine or said prodrug.

In still another embodiment of the present invention, for the treatment of psychotic disorders or conditions in a subjects, the method of the present invention can include administering a triple combination pharmaceutical composition containing an amount of a first therapeutic agent, said first therapeutic agent being ziprasidone;
an amount of a second therapeutic agent, said second therapeutic agent being a benzodiazepine, a prodrug thereof or a pharmaceutically acceptable salt of said benzodiazepine modulator or said prodrug; and
an amount of a third therapeutic agent, said third therapeutic agent being an anticonvulsant, a prodrug thereof or a pharmaceutically acceptable salt of said anticonvulsant or said prodrug.

It will be understood that while the use of a single atypical antipsychotic as a first component compound is preferred, combinations of two or more atypical antipsychotics may be used as a first component if necessary or desired. Similarly, while the use of a single GABA modulator, anticonvulsant or benzodiazepine as a second component compound is preferred, combinations of two or more of these agents may be used as a second component if necessary or desired.

The atypical antipsychotic of the present invention is useful alone or in combination with a second antipsychotic agent, for example, an atypical antipsychotic such as ziprasidone mesylate, a typical antipsychotic such as haloperidol, or a dopamine system stabilizer antipsychotic such as aripiprazole. In addition, the combinations of the present invention may be used in combination with other therapeutic agents for anxiety, i.e. SSRIs, or buspirone or agents for psychotic or mood disorders, i.e. lithium, tricyclic antidepressants. It is preferred that if a second antipsychotic agent is used that they both administered to the patient in synergistic effective amounts. It is preferred that the total amount ranges from about 0.0001 to about 1000 mg/kg per day, more preferably from about 0.01 to about 100 mg/kg per day and most preferably from about 0.1 to about 60 mg/kg per day.

Pharmaceutical compositions of use in the present invention will comprise one or both active compound(s) in association with a pharmaceutically acceptable carrier. Preferably these compositions are in unit dosage forms such as tablets, pills, capsules, powders, granules, sterile parenteral solutions or suspensions, metered aerosol or liquid sprays, drops, ampyules, auto-injector devices or suppositories; for oral, parenteral, intranasal, sublingual or rectal administration, or for administration by inhalation or insufflation. For preparing solid compositions such as tablets, the principal active ingredients are mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof.

When referring to these preformulation compositions as homogeneous, it is meant that the active ingredients is dispersed evenly throughout the composition so that the composition may be readily subdivided-into equally effective unit dosage forms such as tablets, pills and capsules. This solid preformulation composition is then subdivided into unit dosage forms of the type described above containing from 0.1 to about 2000 mg of each of the active ingredients of the present invention. Typical unit dosage forms contain from 1 to 300 mg, for example 1, 2, 5, 10, 25, 50 or 100 mg, of the active ingredient. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

When administered in combination, either as a single or as separate pharmaceutical composition(s), the ziprasidone and the GABA modulator, anticonvulsant or benzodiazepine are presented in a ratio which is consistent with the manifestation of the desired effect. In particular, the ratio by weight of ziprasidone to the GABA receptor modulator will suitably be between 0.001 to 1 and 1000 to 1, and especially between 0.01 to 1 and 100 to 1.

The pharmaceutical combinations may be administered on a regimen of up to 6 times per day, preferably 1 to 4 times per day, especially 2 times per day, and most especially once daily.

As used herein the term “subject” includes animals of economic importance such as bovine, ovine, and porcine animals, especially those that produce meat, as well as domestic animals (e.g. cats and dogs), sports animals (e.g. horses), zoo animals, and humans, the latter being most preferred.

### EXAMPLE 1

A pharmaceutical composition could be prepared by combining ziprasidone with a GABA modulator which is either: (a) gabapentin, (b) pregabalin or (c) lamotrigine in a pharmaceutically acceptable carrier. The composition contains respective amounts of ziprasidone and gabapentin, pregabalin or lamotrigine to deliver on a daily basis between about 20 mg to about 160 mg ziprasidone and between about (a) 100 to 400 mg gabapentin; or (b) 1 to 500 mg pregabalin; or (c) 2 to 200 mg lamotrigine. The composition could be administered to a patient for the treatment of schizophrenia on a daily, twice daily, three times daily, or four times daily basis.

It should be understood that the invention is not limited to the particular embodiments described herein, but that various changes and modifications may be made without departing from the spirit and scope of this novel concept as defined by the following claims.

1. A pharmaceutical composition for use in treating a psychiatric condition selected from the group consisting of a treatment-resistant anxiety disorder, a psychotic disorder or condition, or a mood disorder in a mammal comprising (i) a first therapeutic agent which is an atypical antipsychotic and (ii) a second therapeutic agent selected from the group consisting of GABA modulators, anticonvulsants, and benzodiazepines, wherein the amounts of (i) and (ii) are together effective in treating said psychiatric condition.

2. The pharmaceutical composition of claim 1 where the first therapeutic agent is selected from the group consisting of olanzapine, aripiprazole, clozapine, risperidone, sertindole, quetiapine, amisulpride, asenapine, and ziprasidone or a pharmaceutically acceptable salt or a prodrug thereof or a pharmaceutically acceptable salt of said prodrug; and the second therapeutic agent is selected from the group consisting of muscimol, progabide, riluzole, baclofen, gabapentin, vigabatrin, tiagabine, lamotrigine, pregabalin, topiramate, diazepam, lorazepam, clonazepam, oxazepam, dipotassium chlorazepate, chlorasepate, chlordiazepoxide, mediazepam, flurazepam, clobasam, nitrasepam, flunitrasepam, astazolam, bromazepam, alprazolam, lormetasepam, temazepam, brotizolam, triazolam, chlorodiazepam, halazepam, prazepam, valproate, phenyloin, carbamazepine, felbamate, levetiracetam, zonisamide, methoximide, oxycarbazepine, nemotrizine, ethosuximide, nemotrizine or a pharmaceutically acceptable salt or a prodrug thereof or a pharmaceutically acceptable salt of said prodrug.

3. The pharmaceutical composition of claim 1, wherein the first therapeutic agent is ziprasidone, a prodrug or a pharmaceutically acceptable salt thereof or a pharmaceutically acceptable salt of said prodrug.

4. A method for treating in a mammal in need thereof a disorder selected from treatment-resistant anxiety disorder, a psychotic disorder or condition, or a mood disorder, comprising administering to said mammal
i) an amount of a first therapeutic agent which is an atypical antipsychotic; and
ii) an amount of a second therapeutic agent which is selected from the group consisting of GABA modulators, anticonvulsants, and benzodiazepines, wherein the amounts of (i) and (ii) are together effective in treating said disorder.

5. A method according to claim 4, wherein said method is for treating a treatment-resistant anxiety disorder selected from the group consisting of treatment-resistant obsessive-compulsive disorder, treatment-resistant acute stress disorder, treatment-resistant generalized anxiety disorder, treatment-resistant substance-induced anxiety disorder, and treat-resistant anxiety disorder not otherwise specified.

6. A method according to claim 4, wherein said method is for treating a psychotic disorder or condition is selected from the group consisting of treatment-resistant schizophrenia, treatment-resistant schizophreniform disorder, treatment-resistant schizoaffective disorder, treatment-resistant delusional disorder, treatment-resistant brief psychotic disorder, treatment-resistant shared psychotic disorder, treatment-resistant psychotic disorder due to a medical condition, and treatment-resistant psychotic disorder not otherwise specified.

7. A method according to claim 4, wherein said method is for treating a mood disorder or condition selected from the group consisting of unipolar disorders, bipolar disorders, dysthymic disorder, and cyclothymic disorder.

8. A method according to claim 4, wherein the affliction to be treated is a psychotic disorder or condition.

9. A method according to claim 4, further comprising an amount of a third therapeutic agent which is a benzodiazepine; wherein the amounts (i), (ii) and the benzodiazepine are together effective.

10. The method of claim 1 or 4 wherein the atypical antipsychotic is ziprasidone.

11. The method of claim 1 or 4 wherein the atypical antipsychotic is ziprasidone and said ziprasidone is administered in dosages of about 5 mg to about 460 mg daily.

12. The method of claim 1 or 4 wherein the atypical antipsychotic is ziprasidone and said ziprasidone is administered in dosages of about 20 mg to about 200 mg daily.

13. The method of claim 1 or 4 wherein the atypical antipsychotic is ziprasdione and the administration is oral.

14. The method of claim 1 or 4 wherein the atypical antipsychotic is ziprasidone and the ziprasidone is administered parenterally.

15. The method of claim 1 or 4 wherein the atypical antipsychotic is asenapine or a pharmaceutically acceptable salt thereof.
